(19) United States
(12) Patent Application Publication
(10) Pub. No.: US 2005/0287069 A1
(43) Pub. Date: Dec. 29, 2005

(54) Method of determining performance of an antiperspirant compositions

(76) Inventors: David William Walling, Cincinnati, OH (US); Sascha Maier, Cincinnati, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 11/158,171

(22) Filed: Jun. 21, 2005

Related U.S. Application Data
(60) Provisional application No. 60/581,542, filed on Jun. 21, 2004.

(51) Int. Cl. A61K 49/00; A61K 7/32

(52) U.S. Cl. 424/9.1; 424/65

(57) ABSTRACT

A method of determining performance of anhydrous antiperspirant compositions comprising an antiperspirant active, a liquid carrier, and a structurant. The compositions, processes, and methods of the present invention provide enhanced antiperspirant performance.

### CROSS REFERENCE TO RELATED APPLICATION

This application claims the benefit of U.S. Provisional application Ser. No. 60/581,542 (Case 9678P), filed on Jun. 21, 2004.

### FIELD

The present invention relates to a method of determining performance of anhydrous antiperspirant compositions suitable for topical application to human skin, particularly the axilla.

### BACKGROUND

There are many types of antiperspirant compositions that are commercially available or otherwise known in the antiperspirant art. These products typically contain an antiperspirant active, e.g. zirconium or aluminum salts or combinations thereof, solubilized or dispersed in a suitable liquid carrier, and sufficient structurants to produce the desired solid form in cases of sticks and the desired rheological character in cases of creams.

Antiperspirant compositions are designed to provide effective perspiration and odor control while also being cosmetically acceptable during and after application onto the underarm area of the skin. However, the need still exists for a method of determining performance of anhydrous antiperspirant compositions.

### SUMMARY

The present invention relates to a method of determining performance of anhydrous antiperspirant compositions comprising an antiperspirant active, a liquid carrier, and a structurant. The compositions, processes, and methods of the present invention provide enhanced antiperspirant performance.

### DETAILED DESCRIPTION

While the specification concludes with the claims particularly pointing and distinctly claiming the invention, it is believed that the present invention will be better understood from the following description.

The present invention relates to a method to evaluate the performance of antiperspirant compositions. It has been found that compositions that develop higher red values, that is, a higher a-value on a standard L-a-b colorimetric measurement as detailed below, are better performing products. Also, several formulas have been developed that combine the key ingredients of an antiperspirant active, suitable liquid carrier, and suitable structurant with the appropriate making process to achieve the desired high performing value of greater than 2.00 a-value on the disclosed performance test.

All percentages, parts and ratios are based upon the total weight of the antiperspirant compositions of the present invention and all measurements made are at 25° C., unless otherwise specified. All such weights as they pertain to listed ingredients are based on the active level and, therefore, do not include carriers or by-products that may be included in commercially available materials, unless otherwise specified.

As used herein, the term “antiperspirant compositions” are those compositions that are applied in a thin film to the axilla area in order to reduce or eliminate underarm perspiration. Products contemplated by the phrase “antiperspirant composition” include, but are not limited to, liquids (e.g., aerosols, pump sprays, roll-ons), solids (e.g., gel solids, invisible solids, wax solid sticks), semi-solids (e.g. creams, soft solids, lotions), and the like, provided that the selected form contains all the essential elements as defined

The term “ambient conditions,” as used herein, refers to surrounding conditions under about one atmosphere of pressure, at about 50% relative humidity, and at about 2° C., unless otherwise specified.

The term “anhydrous” as used herein means that the antiperspirant stick composition of the present invention, and the essential or optional components thereof, are substantially free of added or free water. From a formulation standpoint, this means that the anhydrous antiperspirant stick compositions of the present invention contain less than about 1%, and more specifically zero percent, by weight of free or added water, other than the water of hydration typically associated with the particulate antiperspirant active prior to formulation.

The term “structurant”, as used herein, means any material known or otherwise effective in providing suspending, gelling, viscosifying, solidifying and/or thickening properties to the composition, or those materials which otherwise provide structure to the final product form. These solid structurants include one or more solid crystalline or other nonpolymeric suspending agents suitable for topical application to human skin. Suitable suspending agents are those that can form in the composition a crystalline or other matrix within which volatile solvents, non-volatile solvents, or other liquid components of the composition are contained. Such materials will typically be solids under ambient conditions and include organic solids, waxes, crystalline or other gellants, or combinations thereof. A structurant provides a uniform distribution of the particulate active throughout the product and also controls product hardness or rheology.

The term “particulate,” as used herein, refers to compositions or materials that are comprised of solid particles and are not dissolved in water or other solvents.

The term “volatile,” as used herein, unless otherwise specified, refers to those materials that are liquid under ambient conditions and which have a measurable vapor pressure at 25° C. These materials typically have a vapor pressure greater than about 0.01 mmHg, more typically from about 0.02 mmHg to about 20 mmHg, and an average boiling point typically less than about 250° C., more typically less than about 235° C.

The term “cosmetically acceptable”, as used herein, means that the product glides on smoothly during application, is non-irritating, and results in little or no visible residue (e.g., low residue performance) after application to the skin.

I. Composition

A. Antiperspirant Active

The antiperspirant compositions of the present invention comprise a particulate antiperspirant active suitable for application to human skin. The concentration of antiperspirant active in the composition should be sufficient to provide the desired perspiration and odor control from the antiperspirant stick formulation selected.

The anhydrous antiperspirant stick compositions of the present invention comprise an antiperspirant active at concentrations of from about 0.5% to about 60%, and more preferably from about 5% to about 35%, by weight of the composition. These weight percentages are calculated on an anhydrous metal salt basis exclusive of water and any complexing agents such as, for example, glycine and glycine salts. The antiperspirant active as formulated in the composition are in the form of dispersed particulate solids having a preferred average particle size or equivalent diameter of less than about 100 microns, more preferably less than about 20 microns, and even more preferably less than about 10 microns.

The antiperspirant active for use in the anhydrous antiperspirant compositions of the present invention may include any compound, composition or other material having antiperspirant activity. More specifically, the antiperspirant actives may include astringent metallic salts, especially inorganic and organic salts of aluminum, zirconium and zinc, as well as mixtures thereof. Even more specifically, the antiperspirant actives may include aluminum-containing and/or zirconium-containing salts or materials, such as, for example, aluminum halides, aluminum chlorohydrate, aluminum hydroxyhalides, zirconyl oxyhalides, zirconyl hydroxyhalides, and mixtures thereof.

1. Aluminum Salts

Aluminum salts useful in the present invention include those that conform to the formula:
Al₂(OH)ₐ Cl_{b} ·x H₂O

wherein a is from about 2 to about 5; the sum of a and b is about 6; x is from about 1 to about 6; and a, b, and x may have non-integer values. Particularly preferred are the aluminum chlorohydroxides referred to as “5/6 basic chlorohydroxide”, wherein a=5, and “⅔ basic chlorohydroxide”, wherein a=4.

Processes for preparing aluminum salts are disclosed in U.S. Pat. No. 3,887,692, Gilman, issued Jun. 3, 1975; U.S. Pat. No. 3,904,741, Jones et al., issued Sep. 9, 1975; U.S. Pat. No. 4,359,456, Gosling et al., issued Nov. 16, 1982; and British Patent Specification 2,048,229, Fitzgerald et al., published Dec. 10, 1980. Mixtures of aluminum salts are described in British Patent Specification 1,347,950, Shin et al., published Feb. 27, 1974.

2. Zirconium Salts

Preferred zirconium salts for use in the present invention include those which conform to the formula:
ZrO(OH)₂₋ₐ Clₐ·x H₂O

wherein a is from about 1.5 to about 1.87; x is from about 1 to about 7; and a and x may both have non-integer values.

These zirconium salts are described in Belgian Patent 825,146, Schmitz, issued Aug. 4, 1975. Preferred zirconium salts are those complexes that additionally contain aluminum and glycine, commonly known as “ZAG complexes”. These ZAG complexes contain aluminum chlorohydroxide and zirconyl hydroxy chloride conforming to the above-described formulas. Such ZAG complexes are described in U.S. Pat. No. 3,679,068, Luedders et al., issued Feb. 12, 1974; Great Britain Patent Application 2,144,992, Callaghan et al., published Mar. 20, 1985; and U.S. Pat. No. 4,120,948, Shelton, issued Oct. 17, 1978.

B. Anhydrous Liquid Carriers

The antiperspirant compositions of the present invention may comprise an anhydrous liquid carrier at concentrations ranging from about 10% to about 90%, preferably from about 20% to about 80%, more preferably from about 30% to about 70 %, by weight of the composition. Such concentrations will vary depending upon variables such as product form, desired product hardness, selection of other ingredients in the composition, and so forth. The anhydrous liquid carrier for use in the composition can be any anhydrous liquid that is known for use in personal care applications or is otherwise suitable for topical application to the skin. Preferred anhydrous liquid carriers include both volatile fluids and nonvolatile fluids.

1. Volatile Fluid

The antiperspirant composition of the present invention may further comprise a volatile fluid, preferably a volatile silicone carrier at concentrations ranging from about 20% to about 80%, and more specifically from about 30% to about 60%, by weight of the composition. The volatile silicone of the solvent may be cyclic, linear, and/or branched chain silicone. “Volatile silicone”, as used herein, refers to those silicone materials that have measurable vapor pressure under ambient conditions. Non-limiting examples of suitable volatile silicones are described in Todd et al., “Volatile Silicone Fluids for Cosmetics”, Cosmetics and Toiletries, 91:27-32 (1976).

The volatile silicone is preferably a cyclic silicone having from about 3 to about 7 silicone atoms, and more preferably from about 5 to about 6, and still more preferably about 5 silicone atoms. Preferred are those which conform to the formula:

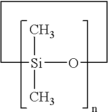

wherein n is from about 3 to about 7, preferably from about 5 to about 6, more preferably about 5. These volatile cyclic silicones generally have a viscosity of less than about 10 centistokes at 25° C. Suitable volatile silicones for use herein include, but are not limited to, Cyclomethicone D5 (commercially available from G. E. Silicones); Dow Corning 344, and Dow Corning 345 (commercially available from Dow Corning Corp.); and GE 7207, GE 7158 and Silicone Fluids SF-1202 and SF-1173 (available from General Electric Co.). SWS-03314, SWS-03400, F-222, F-223, F-250, F-251 (available from SWS Silicones Corp.); Volatile Silicones 7158, 7207, 7349 (available from Union Carbide); Masil SF-V (available from Mazer) and combinations thereof.

2. Non-Volatile Fluid

The antiperspirant composition of the present invention may further comprise a non-volatile fluid. These non-volatile fluids may be either non-volatile organic fluids or non-volatile silicone fluids.
a. Non-Volatile Organic Fluids

The antiperspirant composition of the present invention may further comprise non-volatile organic fluids. The non-volatile organic fluid can be present at concentrations ranging from about 1% to about 20%, and more preferably from about 2% to about 15%, by weight of the composition.

Non-limiting examples of nonvolatile organic fluids include mineral oil, PPG-14 butyl ether, isopropyl myristate, petrolatum, butyl stearate, cetyl octanoate, butyl myristate, myristyl myristate, C12-15 alkylbenzoate (e.g., Finsolv.™.), dipropylene glycol dibenzoate, PPG-15 stearyl ether benzoate and blends thereof (e.g. Finsolv TPP), neopentyl glycol diheptanoate ( e.g. Lexfeel 7 supplied by Inolex), octyldodecanol, isostearyl isostearate, octododecyl benzoate, isostearyl lactate, isostearyl palmitate, isononyl/isononoate, isoeicosane, octyldodecyl neopentanate, hydrogenated polyisobutane, and isobutyl stearate. Many such other carrier liquids are disclosed in U.S. Pat. No. 6,013,248 (Luebbe et al.) and U.S. Pat. No. 5,968,489 (Swaile et al).

b. Nonvolatile Silicone Fluids

The antiperspirant compositions of the present invention may further comprise a non-volatile silicone fluid. The non-volatile silicone fluid is preferably a liquid at or below human skin temperature, or otherwise in liquid form within the anhydrous antiperspirant composition during or shortly after topical application. The concentration of the non-volatile silicone is from about 1% to about 15%, more preferably from about 2% to about 10%, by weight of the composition. Preferred are those nonvolatile silicone fluids which conform to the formula:

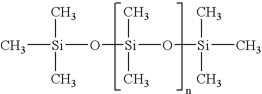

wherein n is greater than or equal to 1. These linear silicone materials will generally have viscosity values of from about 10 centistokes to about 100,000 centistoke, preferably less than about 500 centistoke, more preferably from about 5 centistoke to about 200 centistoke, even more preferably from about 10 centistoke to about 50 centistoke, as measured under ambient conditions.

Specific non limiting examples of suitable nonvolatile silicone fluids include Dow Corning 200, hexamethyldisiloxane, Dow Corning 225, Dow Corning 1732, Dow Corning 5732, Dow Corning 5750 (available from Dow Corning Corp.); and SF-96, SF-1066 and SF18(350) Silicone Fluids (available from G.E. Silicones).

Preferably, the low surface tension non-volatile solvent is selected from the group consisting of dimethicones, dimethicone copolyols, phenyl trimethicones, alkyl dimethicones, alkyl methicones, and mixtures thereof.

C. Structurants

The antiperspirant compositions of the present invention contain a structurant. The structurant may be present in an amount of from about 0.01% to about 25%, more preferably from about 1% to about 15%, even more preferably from about 2.0% to about 10%.

The structurant can be selected from the group consisting of petroleum wax, such as ozokerite or ceresin, stearyl alcohol and other fatty alcohols; hydrogenated castor oil; beeswax; camauba; candelilla; spermeceti wax; baysberry; synthetic waxes, such as Fisher-Tropsch waxes and non-petroleum based microcrystalline wax; polyethylenes with a molecular weight of from about 200 to about 1000 daltons; suitable fatty acid esters such as Syncrowax ERL-C available from Croda, solid triglycerides such as Syncrowax HRC and HGL-C from Croda; and any combination and mixtures thereof. Other non-limiting examples of structurants suitable for use herein are described in U.S. Pat. No. 5,976,514 (Guskey et al.) and U.S. Pat. No. 5,891,424 (Bretzler et al.).

D. Optional Ingredients

The antiperspirant compositions of the present invention may further comprise any optional material that is known for use in antiperspirant and deodorant compositions or other personal care products, or which is otherwise suitable for topical application to human skin. Nonlimiting examples of optional materials include dyes or colorants, emulsifiers, perfumes, distributing agents, antimicrobials, deodorant perfumes, pharmaceutical or other topical actives, preservatives, surfactants, processing aides such as viscosity modifiers, wash-off aids, and so forth. Examples of such optional materials are described in U.S. Pat. No. 4,049,792 (Elsnau); U.S. Pat. No. 5,019,375 (Tanner et al.); and U.S. Pat. No. 5,429,816 (Hofrichter et al.).

II. Process

The antiperspirant compositions of the present invention may be prepared by any known or otherwise effective technique, suitable for providing an anhydrous composition of the desired form and having the essential materials described herein. Many such techniques are described in the antiperspirant/deodorant formulation arts for the described product forms.

For example, the antiperspirant stick compositions can be formulated by mixing the volatile silicone and nonvolatile fluid materials under ambient conditions, or under conditions sufficient to render the admixture fluid or liquid, and then adding the structurants to the mixture and heating the resulting mixture sufficiently to liquefy the added structurants, e.g., at approximately 85° C. for many wax solids, to form a single phase liquid. Antiperspirant solids can then be added to and dispersed throughout the heated, single-phase liquid before allowing the resulting combination to cool to approximately 78° C., at which point perfumes and similar other materials (if any) can be mixed into the combination. The combination can then be cooled to just above the solidification point of the suspending agent (e.g., typically about 60° C.), deposited into dispensing packages, and allowed to solidify under ambient conditions.

III. Test Method

The test method used to determine performance of the antiperspirant product is described below.

A. Preparation of Starting Solutions:

Phenol Red/Deionized Water solution (Deionized Water, 99.985% per weight; Phenol Red, 0.015% per weight) can be prepared as follows: Add Phenol Red powder to deionized water at room temperature. Stir for approximately 2 minutes at approximately 500 rpm, or until the powdered phenol red is completely dissolved into solution, using a magnetic stir bar and stir plate.

Potassium Hydroxide/Deionized Water solution (Deionized Water, 95% per weight; Potassium Hydroxide (solid), 5% per weight) can be prepared as follows: Add Potassium Hydroxide pellets to deionized water at room temperature. Stir for approximately 1 minute at approximately 500 rpm, or until the potassium hydroxide is completely dissolved into solution, using magnetic astir bar and stir plate.

B. Preparation of pH-Indicator Solution:

The pH-indicator solution can be prepared as follows: Weigh 200 grams of above Phenol Red/Deionized Water solution into a glass beaker. At ambient conditions, stir solution continuously at 100 rpm using a magnetic stir bar and stir plate. Insert calibrated pH probe, such as the Orion 8102BNV from Ross, into the solution. Measure pH continuously. Adjust pH of Phenol Red/Deionized Water solution to 10.00+/−0.05 by adding 1 ml increments of Potassium Hydroxide/Deionized Water solution to the beaker containing the phenol red/deionized water solution while continually stirring.

C. Preparation of Antiperspirant Sample:

A film of the antiperspirant sample can be prepared using the following procedure. BYTAC TYPE VF-81 chemical resistant Norton FEP film is cut into 3×7 cm rectangles. A circle 2.2 cm in diameter is punched out. The protective back layer of the film is removed and the sticky side of the BYTAC film is adhered to a standard glass microscope slide. Care is taken such that the 2.2 cm circle cut out of the middle of the film is completely on the microscope slide. Antiperspirant is applied on the microscope slide in the center of the circle cut out of the BYTAC film. The antiperspirant sample is thoroughly spread throughout the circle by using a spatula or equivalent in a back and forth motion across the film surrounding the cut out circle. Spreading is continued until a smooth surface of antiperspirant product across the entire cut out circle is achieved. Carefully remove the BYTAC film from the microscope slide leaving behind the smooth, antiperspirant film. The antiperspirant film on the microscope slide is circular with a thickness equal to the thickness of the just removed BYTAC film (˜0.1778 mm).

D. Application of ph Solution:

The antiperspirant film on the microscope slide is dried for about 24 hours at ambient conditions (first drying period). After the drying period, 20.0 microliters of the phenol red pH-indicator solution are applied to the center of the dried, antiperspirant sample using a standard micropipette such as the 5-50 microliter adjustable Finnpipette from Thermo Labsystems. The sample with the applied 20.0 microliters of phenol red ph indicator solution is left to dry for about 24 hours at ambient conditions (second drying period).

E. Data Collection and Analysis:

After this second 24 hour drying period, the microscope slide with the dried antiperspirant film is placed face up on an approximate 15.24 cm×15.24 cm sample of black felt. A metal ring (1.8 cm diameter×2.5 cm height) is placed on the dried sample eliminating possible contamination of measurements by outside light sources. Care is taken to ensure the dried circle on phenol red ph solution is located in the center of the metal ring. A calibrated Minolta CR-300 series colorimeter, or equivalent, is placed on top of the metal ring. A standard spectral photometric measurement is taken and converted into standard L-a-b scale readings. At least four measurements are taken per sample. The average of the several (at least four) a-value readings is reported.

Preferred anhydrous antiperspirant compositions have an a-value measurement of at least about 2.0, more preferably at least about 2.1, even more preferably, at least about 2.2.

IV. Product Form

The antiperspirant compositions of the present invention can be formulated as any known or otherwise effective product form for providing topical application of antiperspirant or deodorant active to the desired area of the skin. Non-limiting examples of such product forms include liquids (e.g., aerosols, pump sprays, roll-ons), solids (e.g., gel solids, invisible solids, wax solid sticks), semi-solids (e.g. creams, soft solids, lotions), and the like, provided that the selected form contains all the essential elements as defined herein. Preferably, the antiperspirant compositions of the present invention are semi-solids or solids.

The antiperspirant products are generally stored in and dispensed from a suitable package or applicator device, such as a cream dispenser with perforated application domes, etc. These packages should be sufficiently closed to prevent excessive loss of volatiles prior to application.

V. Method of Use

The antiperspirant compositions of the present invention may be applied topically to the underarm or other suitable area of the skin in an amount effective to reduce or inhibit perspiration. Preferably, compositions of the present invention are applied in an amount ranging from at least about 0.1 gram to no more than about 20 grams, preferably no more than about 10 grams, more preferably no more than about 2 grams per axilla.

The composition is preferably applied to the underarm at least about one or two times daily, preferably once daily, to achieve effective antiperspirant reduction or inhibition over an extended period. The antiperspirant composition can also be applied every other day, or every third or fourth day, and then optionally to supplement application on off-days with other personal care products such as deodorants and/or conventional antiperspirant formulations.

Compositions of the present invention are preferably applied to skin, wherein the anhydrous liquid carrier leaves behind a skin-adhering and active-containing film. This film is positioned over the sweat ducts and resists flaking and/or rub-off, thereby being present through multiple perspiration episodes.

VI. Competitive Products

Numerous commercially available antiperspirant products were tested using the above disclosed performance method. The product along with the measured a-value is reported below.

| Product | a-value |
|---|---|
| Old Spice Red Zone Soft Solid | 1.81 |
| Old Spice High Endurance Invisible Solid | 1.45 |
| Old Spice Red Zone Invisible Solid | 1.91 |
| Secret Platinum Soft Solid | 1.8 |
| Secret Platinum Invisible Solid | 1.54 |
| Secret Skin Renewal Invisible Solid | 1.31 |
| Secret Shear Dry | 1.55 |
| Speedstick 24/7 IS | 1.89 |
| Axe IS | 1.4 |
| Dove IS Old Formula | 1.33 |
| Dove IS New Formula | 1.62 |
| Dove White Solid Old Formula | 1.15 |
| Dove White Solid New Formula | 1.59 |
| Gillette Powerstripe IS | 1.04 |
| Rexona Ultra SS | 1.27 |
| Ban IS | 1.89 |
| Ban SS | 0.65 |
| Degree IS | 1.36 |
| Degree SS Tri | 1.17 |
| Degree SS Tetra | 1.6 |
| Suave IS | 1.26 |
| Suave SS | 1.47 |
| Arm & Hammer IS | 1.6 |
| Arrid Total IS | 0.96 |
| Arrid Total SS | 0.84 |
| Mitchum SS | 0.8 |
| Adidas IS | 0.95 |
| Right Guard Powerstripe IS | 1.34 |
| Lady Speedstick IS | 1.4 |

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. The following examples contain a particulate antiperspirant active, a liquid solvent, and a structurant utilizing a method of preparation as detailed above. These compositions are evaluated with the disclosed performance test and have the following results:

| | 1 | 2 | 3 | 4 | 5 | 6 |
|---|---|---|---|---|---|---|
| | wt. % | wt. % | wt. % | wt. % | wt. % | wt. % |
| Aluminum Zirconium | 25.25 | 25.25 | 25.25 | | | |
| Trichlorohydrex | | | | | | |
| Aluminum Zirconium | | | | 25.25 | 25.25 | 25.25 |
| Tetrachlorohydrex | | | | | | |
| Cyclomethicone | 36 | 53.75 | 52.25 | 57.25 | 52.75 | 60.5 |
| Dimethicone | 5 | 5 | 5 | 5 | 5 | 5 |
| Ozokerite Wax | | 13 | 14 | | 14.5 | |
| Syncrowax ERL-C | | 1 | 0.5 | | | |
| Petrolatum | 5 | 2 | 2 | | 2 | 3 |
| Stearyl Alcohol | | | 1 | 1.5 | 0.5 | |
| Polyethylene | 5 | | | 8 | | |
| Glycerol | 7.5 | | | | | 5 |
| Tribehenate | | | | | | |
| Hydrogenated | 7.5 | | | | | |
| Soybean Oil | | | | | | |
| Syncrowax HGL-C | 3.75 | | | | | 1.25 |
| Mineral Oil | 5 | | | 3 | | |
| Performance | 2.04 | 2.09 | 2.09 | 2.2 | 2.31 | 2.43 |
| Test a-value | | | | | | |

### EXAMPLES 1-6

All documents cited in the Background, Summary of the Invention, and Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A method of determining performance of anhydrous antiperspirant compositions, using a phenol red/deionized water solution, a potassium hydroxide/deionized water solution, and a pH-indicator solution, the method comprising the steps of:
a) preparing an antiperspirant sample;
b) applying said pH-indicator solution to said antiperspirant sample;
c) taking a standard spectral photometric measurement of said sample; and
d) converting said measurement into a standard L-a-b scale reading, resulting in an a-value measurement.

2. The method of claim 1, wherein the steps of taking said standard spectral photometric measurement and converting said measurement into standard L-a-b scale reading are repeated at least four times, and the results thereof are averaged.

3. An anhydrous antiperspirant composition having an a-value measurement of at least about 2.0, wherein said composition comprises an antiperspirant active, a liquid carrier, and a structurant, wherein said a-value is measured by a method of determining performance of said composition using a phenol red/deionized water solution, a potassium hydroxide/deionized water solution, and a pH-indicator solution, said method comprising the steps of:
a) preparing an antiperspirant sample;
b) applying said pH-indicator solution to said antiperspirant sample;
c) taking a standard spectral photometric measurement of said sample; and
d) converting said measurement into a standard L-a-b scale reading, resulting in an a-value measurement.

4. The composition of claim 3, wherein said a-value measurement is at least about 2.1.

5. The composition of claim 3, wherein said a-value measurement is at least about 2.2.
